(19) United States
(12) Patent Application Publication
(10) Pub. No.: US 2017/0267976 A1
(43) Pub. Date: Sep. 21, 2017

(54) MEDICAL APPARATUS FOR TRANSFERRING FLUIDS

(71) Applicant: Jennifer Dawn Antoine, Mitchellville, MD (US)

(72) Inventor: Jennifer Dawn Antoine, Mitchellville, MD (US)

(21) Appl. No.: 15/607,454

(22) Filed: May 27, 2017

Related U.S. Application Data
(63) Continuation of application No. 14/756,020, filed on Jul. 23, 2015.

(51) Int. Cl. C12N 5/071; C12N 5/00

(52) U.S. Cl. CPC C12N 5/0676; C12N 2509/00; C12N 5/00

(57) ABSTRACT

A mask is included. The mask includes: a tubing portion having a fastener portion; and a face receptacle portion having a valve portion. The face receptacle portion is coupled to the tubing portion for facilitating the transfer of one or more fluids to a patient. The face receptacle has a semi-filled shape.

### CROSS-REFERENCES TO RELATED APPLICATIONS

The present application is a continuation of pending U.S. patent application entitled, “A Medical Mask for Transferring Fluids,” Ser. No. 14/756,020, filed on Jul. 23, 2015, which is hereby incorporated by reference.

### FIELD OF THE TECHNOLOGY

The present disclosure generally relates to medical devices and, more specifically, to surgical anesthesia masks.

### DESCRIPTION OF THE RELATED ART

Currently, there are 54.1 million surgical procedures performed a year in the U.S. alone. The majority of these surgical procedures use some form of anesthesia for sedating a patient during a surgical procedure. Many of these patients prefer to have the anesthesia administered by inhaling the dosage.

Often, inhaling the anesthesia dosage may be the only option to administer the anesthesia treatment to a patiently given the nature of the injury or the complexity of the surgery. Unfortunately, the anatomy of many patients' faces limits or may prevent effective anesthesia treatment in undertaking a surgical procedure.

Thus, there is a need for a medical device for accommodating the anatomy of a patient's face to administer effective treatment.

### SUMMARY

In one embodiment, a mask is included. The mask includes: a tubing portion having a fastener portion; a face receptacle portion having a valve portion, the face receptacle portion being coupled to the tubing portion for facilitating the transfer of one or more fluids to a patient, wherein the face receptacle portion has a semi-filled shape.

In another embodiment, a medical device is included. The medical device includes one or more tubes having one or more fasteners for connecting the one or more tubes; a receptacle having one or more valves, the receptacle being coupled to the one or more tubes for facilitating the transfer of one or more fluids to a patient, wherein the face receptacle has a semi-filled shape.

In yet another embodiment, an anesthesia mask is included. The anesthesia mask includes one or more tubes having one or more fasteners for connecting the one or more tubes; a face receptacle having one or more valves, the face receptacle being coupled to the one or more tubes for facilitating the transfer of one or more fluids to a patient, wherein the face receptacle has a semi-filled shape.

### BRIEF DESCRIPTION OF THE DRAWINGS

The present invention is described in detail below with reference to the attached drawing figures, which are incorporated by reference herein and wherein:

FIG. 1 is a frontal view of a surgical mask in accordance with an exemplary embodiment of the invention and;

FIG. 2 is a sectional side view of the surgical mask of FIG. 1.

### DETAILED DESCRIPTIONS

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

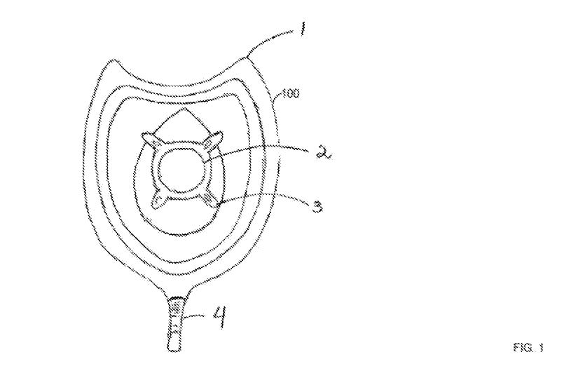

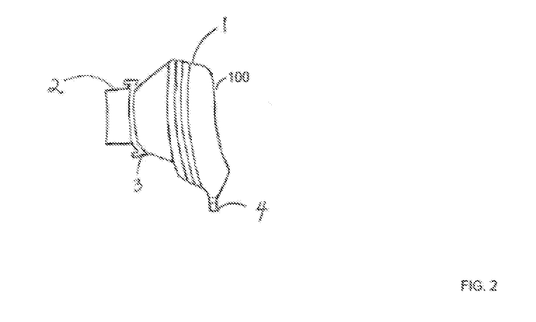

FIGS. 1 and 2 show a surgical mask in accordance with an exemplary embodiment of the invention. As shown in FIGS. 1 and 2, the surgical mask 100 may include a face receptacle portion 1, a tubing portion 2, a fastener portion 3, and a valve portion 4.

In one embodiment, the face receptacle portion 1 may be used to create a tight seal around the mouth of a patient. In yet another embodiment, the face receptacle portion 1 is a configured to be a receiving member or unit for interfacing, touching, or making contacting with portions, segments, or parts of a user's or a patient's face. In some implementations, the face receptacle portion 1 may have a thickness, width, and length in the range of 0.0001 inches to 100 inches.

In some embodiments, the face receptacle portion 1 may include a U-shape, V-shape, or any semi-filled shape design known to those skilled in the art to accommodate portions of a user face such as a nose, mouth, etc. A semi-filled shape may include any shape having a gap, an opening or a portion removed or cut out of the shape. In some implementations, semi filled shape may have a gap portion with a thickness, width, depth, or length in the range of 0.0001 inches to 100 inches. In another embodiment, the face receptacle portion 1 may include a dome shaped or raised portion protruding in the direction of the tubing portion 2. In some embodiments, the dome shaped portion may have a thickness, width, and length in the range of 0.0001 inches to 100 inches.

In some implementations, the tubing portion 2 may be corrugated for attaching a fluid source or dispenser to transfer fluid such as oxygen or aesthesia to a patient. A fluid may include any substance that continually deforms (flows) under an applied shear stress. A fluid may include a subset of the phases of matter and include liquids, gases and plasmas. In an alternate embodiment, the tubing portion 2 may have an extension portion or unit for attaching a hose. In an exemplary embodiment, the extension portion may have a thickness, width, and length in the range of 0.0001 inches to 100 inches. Additionally, in some embodiments, the tubing portion may include one or more tubes. In some implementations, the tubing portion 2 may be partially or completely detachable.

The tubing portion 2 may be made of the following materials: non-toxic polyvinyl chloride (PVC) soft-vinyl, vinyl, plastic, latex, silicone, etc. or known material known by those skilled in the art. The tubing portion 2 may comprise a material composition in the range from 0.00001 to 100%. The diameter of the tubing portion 2 may range from 0.0001 millimeters (mm) to 1000 mm.

In another embodiment, the fastener portion 3 may include one or more fasteners. The fastener portion 3 may include: anchor bolt, batten, brass fastener, buckle, hook, button, cable tie, captive, fastener, clamp (or cramp), hose clamp, clasps, lobster clasp, cleko, clips, circlip, hairpin clip, paper clip, terry clip, clutch, drawing pin (thumbtack), flange, frog, grommet, hook-and-eye closure, hook and loop fastener, latch, nail, pegs, clothespin, tent peg, PEM nut, Pins, bowtie cotter pin, circle cotter, clevis fastener, cotter, dowel, linchpin, R-clip, split pin, spring pin, tapered pin, retaining rings, circlip, e-ring, rivet, rubber band (or bands of other materials), screw anchor, snap fastener, staple, stitches, strap, threaded fastener, captive threaded fasteners, nut, screw, threaded insert, threaded rod, tie, toggle bolt, treasury tag, twist tie, wedge anchor, and zipper or any type of fastener known to those skilled in the art.

In some embodiments, the fastener portion 3 may also be used to connect, attach, or accommodate an external apparatus such as a head strap. In another embodiment, the fastener portion 3 may be partially or completely detachable or removable.

In another embodiment, the valve portion 4 may include a ball valve, butterfly valve, check valve, choke valve, or any type of valve known to those skilled in the art. The valve portion 4 may be used to inflate or deflate the face receptacle portion 1.

In another embodiment, the surgical mask may include an air cushion portion (not shown) for providing a comfortable seal to the skin. In some embodiments, the air cushion may be coupled to the valve portion 4 to inflate or deflate, partially or completely, the cavity of the air cushion. The thickness of the air cushion portion may range from 0.0001 mm to 1000 mm or 0.0001 inches to 100 inches.

In another embodiment, the surgical mask may include a connection port. The connection port (not shown) may be position in the center of the mask for gripping or connecting the tubing portion from a fluid source such as an anesthesia machine.

The surgical mask 100 may be made of the following materials: non-toxic polyvinyl chloride (PVC) soft-vinyl, vinyl, plastic, latex, silicone, etc. or known material known by those skilled in the art. The surgical mask 100 may comprises a material composition in the range from 0.00001 to 100%. In another embodiment, the surgical mask 100 may range in standard anesthesia mask sizes of 1-7. The mask sizes may include a range of 0.0001 inches to 100 inches.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

What is claimed is:

1. A mask comprising:
a tubing portion having a fastener portion;
a face receptacle portion having a valve portion, the face receptacle portion being coupled to the tubing portion for facilitating the transfer of one or more fluids to a patient, wherein the face receptacle portion has a semi-filled, dome shape with a gap between 2.5 inches and 5.5 inches to form a V-shape;
an air cushion for providing for a comfortable seal to the skin, wherein the air cushion has an inflated cavity with a thickness between 1.3 and 1.5 inches; and
the mask is composed of at least three of: silicone, polyvinyl chloride (PVC), latex, and plastic.

2. The mask of claim 1, wherein the fastener portion is a clip.

3. The mask of the claim 1, wherein the semi-filled shape accommodates one or more portions of a face of the patient.

4. The mask of claim 1, wherein the fastener portion is selected from at least one of: a removable hook, a button, and a clasp.

5. The mask of claim 1, further comprising: a distance measured from the top to the bottom of the mask, wherein the distance is between 5.7 inches and 6.4 inches.

6. The mask of claim 1, further comprising: a distance measured from the left side to the right side of the mask, wherein the distance is between 4.1 inches and 5.9 inches.

7. A medical device comprising:
one or more tubes having one or more fasteners for connecting the one or more tubes;
a receptacle having one or more valves, the receptacle being coupled to the one or more tubes for facilitating the transfer of one or more fluids to a patient, wherein the receptacle portion has a semi-filled, dome shape with a gap between 2.5 inches and 5.5 inches to form a V-shape;
an air cushion for providing for a comfortable seal to the skin, wherein the air cushion has an inflated cavity with a thickness between 1.3 and 1.5 inches; and
the medical device is composed of at least three of: silicone, polyvinyl chloride (PVC), latex, and plastic.

8. The medical device of claim 7, wherein the fasteners include a clip.

9. The medical device of claim 7, wherein the semi-filled shape accommodates one or more portions of a face of the patient.

10. The medical device of claim 7, wherein the fasteners are selected from at least one of: a removable hook, a button, and a clasp.

11. The medical device of claim 7, further comprising: a distance measured from the top to the bottom of the medical device, wherein the distance is between 4.8 inches and 5.6 inches.

12. The medical device of claim 7, further comprising: a distance measured from the left side to the right side of the medical device, wherein the distance is between 4.2 inches and 4.6 inches.

13. The medical device of claim 7, further comprising: a distance measured from the top to the bottom of the medical device, wherein the distance is between 4.3 inches and 5.4 inches.

14. The medical device of claim 7, further comprising: a distance measured from the left side to the right side of the medical device, wherein the distance is between 3.9 inches and 4.2 inches.

15. An anesthesia mask comprising:
one or more tubes having one or more fasteners;
a face receptacle having one or more valves, the face receptacle being coupled to the one or more tubes for facilitating the transfer of one or more fluids to a patient, wherein the face receptacle portion has a semi-filled, dome shape with a gap between 2.5 inches and 5.5 inches to form a V-shape;
an air cushion for providing for a comfortable seal to the skin, wherein the air cushion has an inflated cavity with a thickness between 1.3 and 1.5 inches; and
the anesthesia mask is composed of at least three of: silicone, polyvinyl chloride (PVC), latex, and plastic.

16. The anesthesia mask of claim 15, wherein the fasteners include a clip.

17. The anesthesia mask of claim 15, wherein the semi-filled shape accommodates one or more portions of a face of the patient.

18. The anesthesia mask of claim 15, wherein the fasteners are selected from at least one of: a removable hook, a button, and a clasp.

19. The anesthesia mask of claim 15, further comprising: a distance measured from the top to the bottom of the anesthesia mask, wherein the distance is between 4.4 inches and 5.1 inches.

20. The anesthesia mask of claim 15, further comprising: a distance measured from the left side to the right side of the anesthesia mask, wherein the distance is between 3.7 inches and 4.2 inches.
